(19) United States
(12) Patent Application Publication
(10) Pub. No.: US 2007/0117095 A1
(43) Pub. Date: May 24, 2007

(54) Diagnostic method for neonatal or infantile epilepsy syndromes

(76) Inventors: Ingrid Eileen Scheffer, Hawthorn East (AU); Samuel Frank Berkovic, Caulfield North (AU); Sarah Elizabeth Heron, Highbury (AU); John Charles Mulley, Firle (AU)

Correspondence Address: JENKINS, WILSON, TAYLOR & HUNT, P. A., 3100 TOWER BLVD, SUITE 1200, DURHAM, NC, 27707, US

(21) Appl. No.: 10/575,181

(22) PCT Filed: Oct. 13, 2004
(86) PCT No.: PCT/AU04/01399

§ 371 Date: Jan. 30, 2007

(30) Foreign Application Priority Data
Oct. 13, 2003 (AU) 2003905565

(51) Int. Cl. C12Q 1/68

(52) U.S. Cl. 435/6

(57) ABSTRACT

A method for the diagnosis of a neonatal or infantile epilepsy syndrome as BFNIS in a patient comprising testing for the presence of an alteration in the SCN2A gene, including in a regulatory region of the gene, in a patient sample, and establishing a diagnosis which will indicate a high probability of BFNIS when an SCN2A alteration is detected or establishing a diagnosis which will indicate a low probability of BFNIS when an SCN2A alteration is not detected.

### TECHNICAL FIELD

The present invention relates to a diagnostic method for neonatal or infantile epilepsy syndromes.

### BACKGROUND ART

Epilepsies constitute a diverse collection of brain disorders that affect about 3% of the population at some time in their lives (Annegers, 1996). An epileptic seizure can be defined as an episodic change in behaviour caused by the disordered firing of populations of neurons in the central nervous system. This results in varying degrees of involuntary muscle contraction and often a loss of consciousness. Epilepsy syndromes have been classified into more than 40 distinct types based upon characteristic symptoms, types of seizure, cause, age of onset and EEG patterns (Commission on Classification and Terminology of the International League Against Epilepsy, 1989). However the single feature that is common to all syndromes is the persistent increase in neuronal excitability that is both occasionally and unpredictably expressed as a seizure.

A genetic contribution to the aetiology of epilepsy has been estimated to be present in approximately 40% of affected individuals (Gardiner, 2000). As epileptic seizures may be the end-point of a number of molecular aberrations that ultimately disturb neuronal synchrony, the genetic basis for epilepsy is likely to be heterogeneous. There are over 200 Mendelian diseases which include epilepsy as part of the phenotype. In these diseases, seizures are symptomatic of underlying neurological involvement such as disturbances in brain structure or function. In contrast, there are also a number of “pure” epilepsy syndromes in which epilepsy is the sole manifestation in the affected individuals. These are termed idiopathic and account for over 60% of all epilepsy cases.

Idiopathic epilepsies have been further divided into partial and generalized sub-types. Partial (focal or local) epileptic fits arise from localized cortical discharges, so that only certain groups of muscles are involved and consciousness may be retained (Sutton, 1990). However, in generalized epilepsy, EEG discharge shows no focus such that all subcortical regions of the brain are involved. Although the observation that generalized epilepsies are frequently inherited is understandable, the mechanism by which genetic defects, presumably expressed constitutively in the brain, give rise to partial seizures is less clear. In neonates and infants, probably because brain myelination is incomplete, the distinction between generalized and partial epilepsies is less clear from clinical and neurobiological standpoints.

Epilepsies in the first year of life were previously viewed as largely due to acquired peri-natal factors. However, two benign autosomal dominant epilepsy syndromes are now well recognised in the first year of life. The first is benign familial neonatal seizures (BFNS) which usually presents around the third day of life and is characterised by tonic or clonic seizures. These seizures stop within a few weeks of age, with 5% of individuals having later febrile seizures and 11% later epilepsy (Plouin, 1994). Studies have shown that the genetic basis for this syndrome in many cases is due to mutations in the potassium channel genes KCNQ2 and KCNQ3.

The second is benign familial infantile seizures (BFIS) which presents between 4 and 8 months of age, with clusters of tonic or clonic partial or generalised seizures over a few days. Seizures usually resolve by around 1 year of age but it may be associated with paroxysmal dyskinesias in later childhood in some individuals. While no genes have been definitively identified to be causative of BFIS, linkage to chromosomes 19 and 16 have been reported (Szepetowski et al., 1997; Guipponi et al., 1997).

In 1983, prior to the recognition of BFIS, an American family was reported that had an intermediate variant of BFNS and BFIS, termed benign familial neonatal-infantile seizures (BFNIS), where seizure onset varied from 2 days to 3.5 months (Kaplan and Lacey, 1983).

Recently, genetic analysis of two BFNIS families lead to the identification of two mutations in the SCN2A gene that were responsible for the disorder (Heron et al, 2002).

The inventors have built on this study through the analysis of affected individuals from additional families with probable or possible BFNIS. This has lead to the identification of further missense mutations in SCN2A in 6 families that result in changes in evolutionary conserved amino acids. Both families clinically recognised as probable BFNIS and four of nine families recognised as possible BFNIS contained SCN2A mutations. This further emphasizes the importance of genetic factors in epilepsies of the neonatal and early infantile periods. Of 95 families with other forms of childhood epilepsy tested, none contained mutations in SCN2A.

The inventors have established a method for the diagnosis of BFNIS and other neonatal and infantile epilepsies, based on testing for the presence of alterations in the SCN2A, and, optionally, the KCNQ2 and/or KCNQ3 genes, in affected patients. The development of a molecular diagnostic test strategy to aid in the diagnosis of neonatal and infantile epilepsies is important. Such a test strategy enables proper management of the affected patient and avoids over-investigation and over-treatment of the patient.

### DISCLOSURE OF THE INVENTION

In a first aspect of the present invention there is provided a method for the diagnosis of a neonatal or infantile epilepsy syndrome as BFNIS in a patient with seizure onset in the first year of life, comprising testing for the presence of an alteration in the SCN2A gene, including in a regulatory region of the gene, in a patient sample, and establishing a diagnosis which will indicate a high probability of BFNIS when an SCN2A alteration is detected or establishing a diagnosis which will indicate a low probability of BFNIS when an SCN2A alteration is not detected.

This information is important for initiating the correct treatment regimen for a patient and avoids unnecessary testing and associated trauma to the patient.

The nature of the alterations in the SCN2A gene may encompass all forms of gene sequence variations including deletions, insertions, rearrangements and point mutations in the coding and non-coding regions such as the promoter, introns or untranslated regions but, in particular, missense mutations have been associated with BFNIS. Deletions may be of the entire gene or only a portion of the gene whereas point mutations may result in stop codons, frameshifts or amino acid substitutions. Point mutations occurring in the regulatory regions of SCN2A, such as in the promoter, may lead to loss or a decrease of expression of the mRNA or may abolish proper mRNA processing leading to a decrease in mRNA stability or translation efficiency.

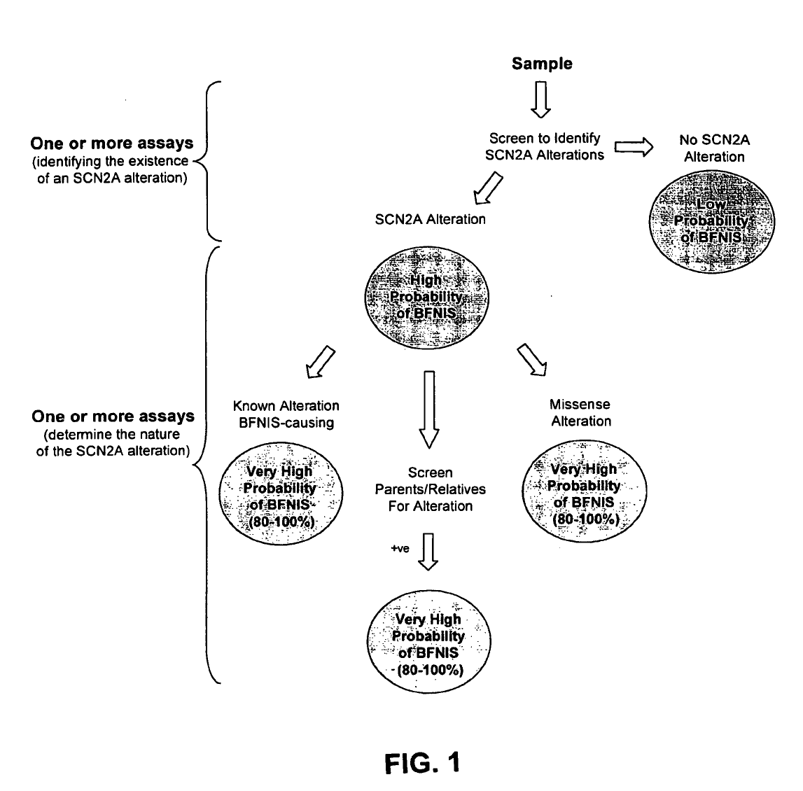

The identification of SCN2A alterations in a patient that have previously been associated with BFNIS, or which are present in the patient's affected parent or relatives increases the likelihood that the patient has BFNIS. Furthermore, information concerning the age of onset may be used to suggest a diagnosis of BFIS or BFNS once BFNIS is ruled out through failure to identify an SCN2A alteration. The flow chart in FIG. 1 illustrates an embodiment of the present invention.

In an embodiment of the invention there is provided a method for the diagnosis of a neonatal or infantile epilepsy syndrome as BFNIS in a patient comprising performing one or more assays to test for the presence of an SCN2A alteration and to identify the nature of the alteration.

In a further embodiment there is provided a method for the diagnosis of a neonatal or infantile epilepsy syndrome as BFNIS in a patient comprising the steps of:
(1) performing one or more assays to test for the presence of an alteration in the SCN2A gene of the patient; and, if the results indicate the presence of an alteration in the SCN2A gene,
(2) performing one or more assays to identify the nature of the SCN2A alteration.

In a further aspect of the invention there is provided a method for the diagnosis of a neonatal or infantile epilepsy syndrome as one of BFNIS, BFNS or BFIS in a patient with seizure onset in the first year of life comprising:
(1) (a) testing for the presence of an alteration in the SCN2A gene, including in a regulatory region of the gene, in a patient sample; and/or
(b) testing for the presence of an alteration in the KCNQ2 and/or KCNQ3 genes, including in regulatory regions of the genes, in the patient sample; and
(2) (a) establishing a diagnosis which will indicate a high probability of BFNIS when an SCN2A alteration is detected;
(b) establishing a diagnosis which will indicate a high probability of BFNS when a KCNQ2 or KCNQ3 alteration is detected; or
(c) establishing a diagnosis which will indicate a likelihood of BFIS when an SCN2A, KCNQ2 or KCNQ3 alteration is not detected.

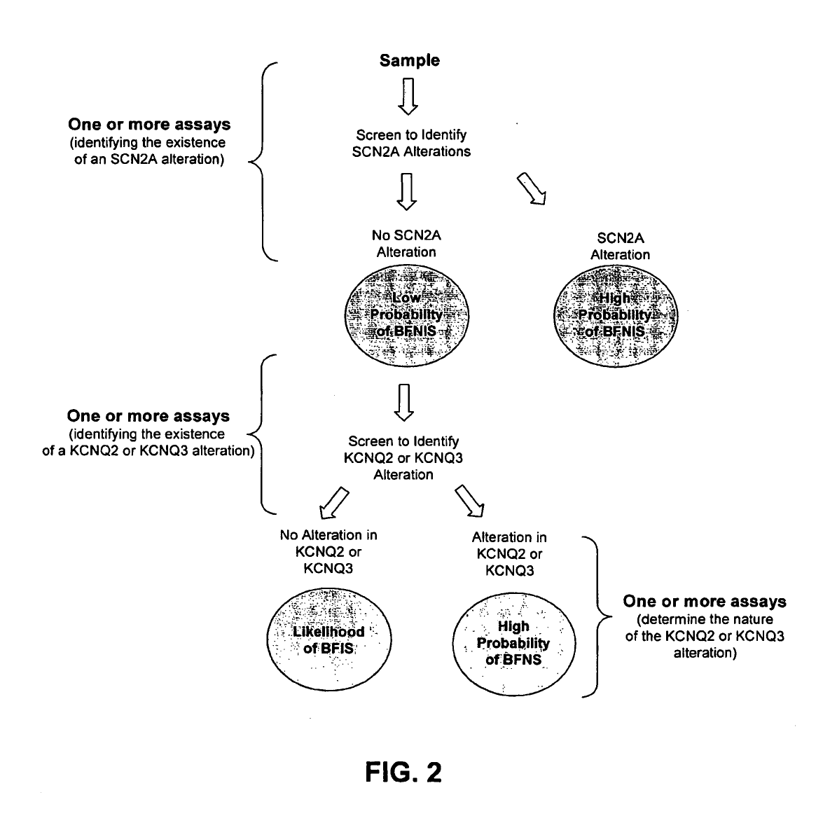

The flow chart in FIG. 2 illustrates an embodiment of the present invention. It will be appreciated that screens to detect alterations in the various subunits may be undertaken in a different order to what is illustrated and yet ultimately provide the same clinical information. For example, a screen for alterations in KCNQ2 could be undertaken followed, if negative, by a screen for alterations in KCNQ3 followed, if negative, by a screen for alterations in SCN2A. Clinical observations involving family history and clinical observations may be employed in determining the order of the screens, and may also be employed in reaching a diagnosis, particularly in reaching a diagnosis of BFIS following negative genetic tests.

The nature of the alterations in the KCNQ2 and KCNQ3 genes may encompass all forms of gene sequence variations as described above for the SCN2A gene.

In a further embodiment of the invention there is provided a method for the diagnosis of a neonatal or infantile epilepsy syndrome as BFNIS, BFNS or BFIS in a patient comprising performing one or more assays to test for the presence of an SCN2A, KCNQ2 or KCNQ3 alteration and to identify the nature of the alteration.

In a further embodiment there is provided a method for the diagnosis of a neonatal or infantile epilepsy syndrome as BFNIS, BFNS or BFIS in a patient comprising the steps of:

(1) performing one or more assays to test for the presence of an alteration in the SCN2A, KCNQ2 or KCNQ3 gene of the patient; and, if the results indicate the presence of an alteration in any one of these genes,

(2) performing one or more assays to identify the nature of the alteration.

There exists a number of assay systems that can be used to test for the presence of SCN2A, KCNQ2 or KCNQ3 alterations and the invention is not limited by the examples that are provided below.

In one embodiment an assay system employed may be the analysis of SCN2A, KCNQ2 or KCNQ3 DNA from a patient sample in comparison to wild-type SCN2A, KCNQ2 or KCNQ3 DNA. Genomic DNA may be used for the diagnostic analysis and may be obtained from a number of sources including, but not limited to, body cells, such as those present in the blood or cheek, tissue biopsy, surgical specimen, or autopsy material. The DNA may be isolated and used directly for the diagnostic assays or may be amplified by the polymerase chain reaction (PCR) prior to analysis. Similarly, RNA or cDNA may also be used, with or without PCR amplification. In addition, prenatal diagnosis can be accomplished by testing fetal cells, placental cells or amniotic fluid.

In a specific embodiment, a DNA hybridisation assay may be employed. These may consist of probe-based assays specific for the SCN2A, KCNQ2 or KCNQ3 genes. One such assay may look at a series of Southern blots of DNA that has been digested with one or more restriction enzymes. Each blot may contain a series of normal individuals and a series of patient samples. Samples displaying hybridisation fragments that differ in length from normal DNA when probed with sequences near or including the SCN2A, KCNQ2 or KCNQ3 genes (SCN2A, KCNQ2 or KCNQ3 gene probes) indicate a possible SCN2A, KCNQ2 or KCNQ3 alteration. If restriction enzymes that produce very large restriction fragments are used then pulsed field gel electropheresis (PFGE) may be employed.

SCN2A, KCNQ2 or KCNQ3 exon specific hybridisation assays may also be employed. This type of probe-based assay will utilize at least one probe which specifically and selectively hybridises to an exon of the SCN2A, KCNQ2 or KCNQ3 gene in its wild-type form. Thus, the lack of formation of a duplex nucleic acid hybrid containing the nucleic acid probe is indicative of the presence of an alteration in the relevant gene. Because of the high specificity of probe-based tests, any negative result is highly indicative of the presence of an alteration however further investigational assays should be employed to identify the nature of the alteration to determine the likelihood it is disease-associated.

The exon specific assay approach could also be adapted to identify previously determined SCN2A, KCNQ2 or KCNQ3 alterations responsible for BFNIS or BFNS. In this aspect, a probe which specifically and selectively hybridises with any one of the SCN2A, KCNQ2 or KCNQ3 genes in its altered form is used (allele specific probe). In this case the formation of a duplex nucleic acid hybrid containing the nucleic acid probe is indicative of the presence of the alteration in the relevant gene. In each variation of the exon specific assay approach, it is important to take into account known polymorphisms in the genes that are not associated with disease. A secondary assay such as DNA sequencing should subsequently be employed to ensure that any suspected alterations are not known polymorphisms.

The exon specific probes used for each of the abovementioned assays may be derived from: (1) PCR amplification of each exon of the SCN2A, KCNQ2 or KCNQ3 genes using intron specific primers flanking each exon; (2) cDNA probes specific for each exon; or (3) a series of oligonucleotides that collectively represent a SCN2A, KCNQ2 or KCNQ3 exon.

In a further embodiment, an assay to analyse heteroduplex formation may be employed. By mixing denatured wild-type SCN2A, KCNQ2 or KCNQ3 DNA with a DNA sample from a patient, any sequence variations between the two samples in the relevant gene being tested will lead to the formation of a mixed population of heteroduplexes and homoduplexes during reannealing of the DNA. Analysis of this mixed population can be achieved through the use of such techniques as high performance liquid chromatography (HPLC) which are performed under partially denaturing temperatures. In this manner, heteroduplexes will elute from the HPLC column earlier than the homoduplexes because of their reduced melting temperature.

In a further embodiment, patient samples may be subject to electrophoretic-based assays. For example electrophoretic assays that determine SCN2A, KCNQ2 or KCNQ3 fragment length differences may be employed.

Fragments of each patient's genomic DNA are amplified with intron specific primers to the relevant gene under investigation. The amplified regions of the gene therefore include the exon of interest, the splice site junction at the exon/intron boundaries, and a short portion of intron at either end of the amplification product. The amplification products may be run on an electrophoresis size-separation gel and the lengths of the amplified fragments are compared to known and expected standard lengths from the wild-type gene to determine if an insertion or deletion mutation is found in the patient sample. This procedure can advantageously be used in a “multiplexed” format, in which primers for a plurality of exons (generally from 2 to 8) are co-amplified, and evaluated simultaneously on a single electrophoretic gel. This is made possible by careful selection of the primers for each exon of the gene. The amplified fragments spanning each exon are designed to be of different sizes and therefore distinguishable on an electrophoresis/size separation gel. The use of this technique has the advantage of detecting both normal and mutant alleles in heterozygous individuals. Furthermore, through the use of multiplexing it can be very cost effective.

In a further approach, diagnostic electrophoretic assays for the detection of previously identified SCN2A, KCNQ2 or KCNQ3 alterations responsible for BFNIS or BFNS may utilise PCR primers which bind specifically to altered exons of the genes. In this case, product will only be observed in the electrophoresis gel if hybridization of the primer occurred. Thus, the appearance of amplification product is an indicator of the presence of the alteration, while the length of the amplification product may indicate the presence of additional alterations.

Additional electrophoretic assays may be employed. These may include the single-stranded conformational polymorphism (SSCP) procedure (Orita et al., 1989). As mentioned above, fragments of each patient's genomic DNA are PCR amplified with intron specific primers to any one of the SCN2A, KCNQ2 or KCNQ3 genes such that individual exons of the genes are amplified and may be analysed individually. Exon-specific PCR products are then subjected to electrophoresis on non-denaturing polyacrylamide gels such that DNA fragments migrate through the gel based on their conformation as dictated by their sequence composition. Exon-specific fragments that vary in sequence from wild-type sequence will have a different secondary structure conformation and therefore migrate differently through the gel. Aberrantly migrating PCR products in patient samples are indicative of an alteration in the exon and should be analysed further in secondary assays such as DNA sequencing to identify the nature of the alteration.

Additional electrophoretic assays that may be employed include RNase protection assays (Finkelstein et al., 1990; Kinszler et al., 1991) and denaturing gradient gel electrophoresis (DGGE)(Wartell et al., 1990; Sheffield et al., 1989). RNase protection involves cleavage of a mutant polynucleotide into two or more smaller fragments whereas DGGE detects differences in migration rates of mutant sequences compared to wild-type sequences, using a denaturing gradient gel.

In the RNase protection assay a labelled riboprobe which is complementary to the human wild-type SCN2A, KCNQ2 or KCNQ3 gene coding sequence is hybridized with either mRNA or DNA isolated from the patient and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full length duplex RNA for the riboprobe and the mRNA or DNA. The riboprobe need not be the full length of the mRNA or gene under investigation but can be a segment of either. If the riboprobe comprises only a segment of the mRNA or gene, it will be desirable to use a number of these probes to screen the whole mRNA sequence for mismatches.

In a further embodiment, enzymatic based assays (Taylor and Deeble, 1999) may be used in diagnostic applications. Such assays include the use of S1 nuclease, ribonuclease, T4 endonuclease VII, MutS (Modrich, 1991), Cleavase and MutY. In the MutS assay, the protein binds only to sequences that contain a nucleotide mismatch in a heteroduplex between mutant and wild-type sequences.

When an assay is to be based upon the SCN2A, KCNQ2 or KCNQ3 protein, a variety of approaches are possible. For example, diagnosis can be achieved by monitoring differences in the electrophoretic mobility of wild-type SCN2A, KCNQ2 or KCNQ3 protein and SCN2A, KCNQ2 or KCNQ3 protein isolated from a patient sample. Such an approach will be particularly useful in identifying alterations in which charge substitutions are present, or in which insertions, deletions or substitutions have resulted in a significant change in the electrophoretic migration of the resultant protein. Alternatively, diagnosis may be based upon differences in the proteolytic cleavage patterns of normal and altered proteins, differences in molar ratios of the various amino acid residues, or by functional assays demonstrating altered function of the gene products.

Further assays that are based on the SCN2A, KCNQ2 or KCNQ3 protein include immunoassays. The procedures for raising antibodies against specific gene products are well described in the literature, for example in U.S. Pat. Nos. 4,172,124 and 4,474,893 which are incorporated herein by reference. Antibodies are normally raised which bind to portions of the gene product away from common mutation sites such that the same antibody binds to both mutant and normal protein. Preferred antibodies for use in this invention are monoclonal antibodies because of their improved predictability and specificity. It will be appreciated, however, that essentially any antibody which possesses the desired high level of specificity can be used, and that optimization to achieve high sensitivity is not required.

For the diagnostic detection of SCN2A, KCNQ2 or KCNQ3 alterations previously identified to be involved in neonatal or infantile epilepsies including BFNIS, BFNS and BFIS, antibody raised against the defective gene product is preferable. Antibodies are added to a portion of the patient sample under conditions where an immunological reaction can occur, and the sample is then evaluated to see if such a reaction has occurred. The specific method for carrying out this evaluation is not critical and may include enzyme-linked immunosorbant assays (ELISA), described in U.S. Pat. No. 4,016,043, which is incorporated herein by reference; fluorescent enzyme immunoassay (FEIA or ELFA), which is similar to ELISA, except that a fluoregenic enzyme substrate such as 4-methylumbelliferyl-beta-galactoside is used instead of a chromogenic substrate, and radioimmunoassay (RIA).

The most definitive diagnostic assay that may be employed is DNA sequencing, and ultimately may be the only assay that is needed to be performed. Comparison of the SCN2A, KCNQ2 or KCNQ3 DNA wild-type sequence with the SCN2A, KCNQ2 or KCNQ3 sequence of a test patient provides both high specificity and high sensitivity. The general methodology employed involves amplifying (for example with PCR) the DNA fragments of interest from patient DNA; combining the amplified DNA with a sequencing primer which may be the same as or different from the amplification primers; extending the sequencing primer in the presence of normal nucleotide (A, C, G, and T) and a chain-terminating nucleotide, such as a dideoxynucleotide, which prevents further extension of the primer once incorporated; and analyzing the product for the length of the extended fragments obtained. While such methods, which are based on the original dideoxysequencing method disclosed by Sanger et al., 1977 are useful in the present invention, the final assay is not limited to such methods. For example, other methods for determining the sequence of the gene of interest, or a portion thereof, may also be employed. Alternative methods include those described by Maxam and Gilbert (1977) and variations of the dideoxy method and methods which do not rely on chain-terminating nucleotides at all such as that disclosed in U.S. Pat. No. 4,971,903, which is incorporated herein by reference. Any sequence differences (other than benign polymorphisms) in exons of a test patient when compared to that of the wild-type sequence indicate a potential disease-causing alteration.

In specific embodiments of the invention, there is provided a method for testing patients for BFNIS-associated mutations in the SCN2A gene comprising the steps of:
a) quantitatively amplifying at least one exon of the SCN2A gene from a body sample of each patient to produce amplified fragments;
b) comparing the properties of the amplified fragments to standard values based upon the fragments produced by amplification of the same exon in a non-mutant SCN2A gene; and
c) determining the nucleic acid sequence of each exon identified in b) that has different properties in the patient compared to the corresponding non-mutant SCN2A exon.

In further specific embodiments there is provided a method for testing patients for BFNIS-associated mutations in the SCN2A gene comprising the steps of:
a) quantitatively amplifying, from a body sample of each patient at least one exon of the SCN2A gene using primers complementary to intron regions flanking each amplified exon;
b) comparing the length of the amplification products for each amplified exon to the length of the amplification products obtained when a wild-type SCN2A gene is amplified using the same primers, whereby differences in length between an amplified sample exon and the corresponding amplified wild-type exon reflect the occurrence of a truncating mutation in the sample SCN2A gene; and
c) determining the nucleic acid sequence of each exon identified in b) to contain a truncating mutation.

In even further specific embodiments there is provided a method for testing patients for BFNIS-associated mutations in the SCN2A gene comprising the steps of:
a) quantitatively amplifying, from a body sample of each patient at least one exon of the SCN2A gene using primers complementary to intron regions flanking each amplified exon;
b) hybridising the fragments from a) with fragments produced by amplification of the same exon in a non-mutant SCN2A gene;
c) determining the nucleic acid sequence of each patient exon identified in b) that either does not hybridise to corresponding wild-type fragments or forms a mismatched heteroduplex.

Throughout this specification and the claims, the words “comprise”, “comprises” and “comprising” are used in a non-exclusive sense, except where the context requires otherwise.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

### MODES FOR PERFORMING THE INVENTION

Prior to the current study, mutations in the SCN2A gene were seen in 2 families with BFNIS. This finding has been expanded upon by the analysis of additional families with early childhood epilepsies so as to refine the molecular-clinical correlation of SCN2A mutations in these epilepsy types.

### Example 1

### SCN2A Mutation Analysis in Neonatal and Infantile Epilepsies

The current study examined three sets of subjects for SCN2A mutations using SSCP analysis and sequencing. These included 2 families with probable BFNIS based on a clinical assessment, nine families with possible BFNIS based on the fact that most individuals had seizures before 4 months of age and in some families neonatal seizures were observed, and 103 additional families constituting other early childhood epilepsies. In these 103 families, 10 had BFIS, 59 had generalised epilepsy with febrile seizures plus (GEFS+) in whom mutations in SCN1A, SCN1B and GABRG2 were not detected, and 32 constituted unrelated cases with benign childhood epilepsy with centrotemporal spikes.

The results of the mutation analysis of SCN2A in these families showed that missense mutations resulting in changes in evolutionary conserved amino acids were found in a total 6 families. Both families categorized as probable BFNIS, and four of the nine families regarded as possible BFNIS were positive. The mutations in these families were not found in the controls.

No mutations were found in the 59 GEFS+families. Neither of the two families with exclusively neonatal onset had SCN2A mutations. Of the 10 families with BFIS, an A1822V change was seen in two members of one family, while one child from the benign childhood epilepsy with centrotemporal spikes group had a T1200A alteration in SCN2A that was not seen in the normal population.

From the current work, the clinical and molecular characterisation of additional families with SCN2A mutations now establishes BFNIS as an important sodium channelopathy of the early infantile period (Berkovic et al., 2004).

### Example 2

### Diagnostic Method—Assay System Examples

Based on the findings of this study, a method for the diagnosis of BFNIS, BFNS or BFIS in a patient has been established. The flowchart in FIG. 1 illustrates a strategy based on the invention that can be used to determine the likelihood that an alteration in the SCN2A gene is responsible for BFNIS, and further to make a diagnosis of BFNS or BFIS. In addition, the flowchart in FIG. 2 illustrates a molecular biology-based strategy that can be used to establish the likelihood that a neonatal or infantile seizure patient has BFNIS, BFNS or BFIS. This is based on the fact that BFNIS is associated with SCN2A alterations, BFNS is associated with KCNQ2 or KCNQ3 alterations and BFIS is not associated with alterations in any of these genes.

The assay combination chosen for the diagnostic method is preceded by selecting the patient population to be examined and obtaining DNA from the sample population. The sample population may encompass any individual with epilepsy but would likely focus on patients where seizure onset is before 6 months of age.

DNA from a test patient may be obtained in a number of ways. The most common approach is to obtain DNA from blood samples taken from the patient, however DNA may also be obtained using less invasive approaches such as from cheek cell swabs.

For the current study DNA was extracted from collected blood using the QIAamp DNA Blood Maxi kit (Qiagen) according to manufacturer's specifications or through procedures adapted from Wyman and White (1980). For DNA samples obtained using the QIAamp kit, a final ethanol precipitation step was employed with DNA pellets being resuspended in sterile water. Stock DNA samples were kept at a concentration of 200 ng/ul and 100 ng/ul dilutions were prepared for subsequent PCR reactions.

Any combination of assay systems described above may be employed using the method. Provided below are examples of assays that were employed for the detection of alterations in the SCN2A gene and a determination of their nature in the present study. Assays that may be employed for the detection of alterations in KCNQ2 and/or KCNQ3 are described in WO99/21875, the contents of which are incorporated herein by reference.

SCN2A Electrophoretic Assay Using SSCP—Identifying the Existence of an SCN2A Alteration

Once DNA from a patient had been obtained, PCR amplification of individual exons of the SCN2A gene was employed prior to analysis by single strand conformation polymorphism (SSCP) analysis. Table 1 provides a list of primers that may be employed to analyse each exon of the SCN2A gene and which were used in the present study.

In this specific example, primers used for SSCP were labelled at their 5′ end with HEX and typical PCR reactions were performed in a total volume of 10 μl. All PCR reactions contained 67 mM Tris-HCl (pH 8.8); 16.5 mM (NH₄)₂SO₄; 6.5 μM EDTA; 1.5 mM MgCl₂; 200 μM each dNTP; 10% DMSO; 0.17 mg/ml BSA; 10 mM β-mercaptoethanol; 5 μg/ml each primer and 100 U/ml Taq DNA polymerase. PCR reactions were typically performed using 10 cycles of 94° C. for 30 seconds, 60° C. for 30 seconds, and 72° C. for 30 seconds followed by 25 cycles of 94° C. for 30 seconds, 55° C. for 30 seconds, and 72° C. for 30 seconds. A final extension reaction for 10 minutes at 72° C. followed.

Ten to twenty μl of loading dye comprising 50% (v/v) formamide, 12.5 mM EDTA and 0.02% (w/v) bromophenol blue were added to completed reactions which were subsequently run on non-denaturing 4% polyacrylamide gels with a cross-linking ratio of 35:1 (acrylamide:bis-acrylamide) and containing 2% glycerol. Gel thickness was 100 μm, width 168 mm and length 160 mm. Gels were run at 1200 volts and approximately 20 mA, at 18° C. and analysed on the GelScan 2000 system (Corbett Research, Australia) according to manufacturers specifications.

DNA Sequencing Assay—Identifying the Nature of an SCN2A Alteration

PCR products from the SSCP analysis that showed a conformational change were subject to secondary assays such as DNA sequencing to determine the nature of the change. In the example provided here, this first involved re-amplification of the amplicon displaying a band-shift from the relevant patient (primers used in this instance did not contain 5′ HEX labels) followed by purification of the PCR amplified templates for sequencing using QiaQuick PCR preps (Qiagen) based on manufacturer's procedures. The primers used to sequence the purified amplicons were identical to those used for the initial amplification step. For each sequencing reaction, 25 ng of primer and 100 ng of purified PCR template were used. The BigDye sequencing kit (ABI) was used for all sequencing reactions according to the manufacturer's specifications. The products were run on an ABI 377 Sequencer and analysed using the EditView program.

A comparison of the DNA sequence obtained from the patient sample can then be made directly to that of the wild-type SCN2A sequence in order to detect the DNA alteration that lead to the conformational change detected by SSCP. If the DNA change is not a known polymorphism in the SCN2A gene, it is likely that it may be a disease causing mutation essentially providing a diagnosis that can be investigated further through the analysis of additional family members.

Additional Assays—dHPLC Assay

In addition to the assays described above, other assays may be employed to test for the existence of alterations in the SCN2A gene that are associated with BFNIS. One such assay is high performance liquid chromatography (dHPLC). In this technique, DNA obtained from the patient is first PCR amplified for individual exons of the SCN2A gene. The primers employed for SSCP analysis (see Table 1) may also be used for dHPLC analysis.

dHPLC PCR reactions and cycling conditions can be performed as described above for SSCP analysis, however any PCR reaction and cycling conditions may be employed provided that the amplification produces a distinct product specific for the amplicon under investigation only.

An example of alternative PCR reaction conditions are where the reaction is performed in a total volume of 20 μl containing 1×PCR buffer (Invitrogen), 200 uM dNTPs, 300 ng of each primer, 1.5 mM MgCl₂, 100 ng DNA and 0.5 units of Taq DNA polymerase (Invitrogen). PCR cycling conditions will vary depending on the nature of the amplicon and primer sequence but typically may involve 1 cycle of 94° C. for 2 minutes, followed by 10 cycles of 60° C. for 30 seconds, 72° C. for 30 seconds, and 94° C. for 30 seconds, followed by 25 cycles of 55° C. for 30 seconds, 72° C. for 30 seconds, and 94° C. for 30 seconds. A final annealing reaction at 55° C. for 30 seconds followed by an extension reaction for 10 minutes at 72° C. usually completes the cycling.

Prior to dHPLC analysis, PCR products are heated to 95° C. for 5 minutes and are then slowly cooled at −3° C. increments for 1.5 minutes (until 25° C. is reached). This is to allow the formation of hetero-and homoduplexes depending upon the nucleotide constitution of the PCR product.

Various dHPLC systems can be used for heteroduplex analysis and mutation detection. One example is the Transgenomic WAVE® System. In order to detect mutations on the dHPLC each product is required to be run under partially denaturing conditions. Due to each amplicon of the SCN2A gene having a different sequence, the temperature(s) at which each product is partially denatured needs to be first calculated.

Amplicons are fed through the dHPLC column and computer generated chromatograms are compared between patient samples and wild-type samples. The analysis may be done by visual inspection of the chromatograms or, in the case of the Transgenomic WAVE® System, using software supplied with the system. Those patient samples showing different peak patterns to wild-type are considered to contain alterations in the SCN2A amplicon under investigation and the DNA from those individuals can be subjected to a further assay, namely DNA sequencing (see example above), to determine the nature of the SCN2A alteration and to predict the likelihood that the alteration is responsible for BFNIS.

TABLE 1
Primer Sequences Used for Mutation Analysis of SCN2A

| | | | Size |
|---|---|---|---|
| Exon | Forward Primer | Reverse Primer | (bp) |
| 5′ UTR | ACAGGAAGTTAGGTGTGGTC | GAGAAGCATCACAGAG | 206 |
| 1a | TGCTGTATCTCAGTGCTCAG | TCATCATCCTCATCCTTGCG | 281 |
| 1b | GCTAAGAGACCCAAAC | TAGGCAGTGAAGGCAACTTG | 201 |
| 2 | GGCACTATTTTACAGGGC | CATAACATTGCCAACCACAG | 325 |
| 3 | TGGTGAAGGCATGGTAGT | ATTGAGGAGGTCTCAAGGTG | 239 |
| 4 | ACCAACCTGGAAGTGTCT | ATAGTATAGGCTCCCACCAG | 300 |
| 5 | AGGCCCCTTATATCTCCAAC | TAGCAACAAGGCTTCTGCAC | 244 |
| 5n | GATGAAAGACCAAGGAAGAC | TGGAGATATAAGGGGCCTAG | 200 |
| 6a | TTCCAGGACAAGCTCATG | GGAAGAATTATCTGGAGGCCA | 249 |
| 6b | TTGTTCATGGGCAACCTACG | GTCTAAGTCACTTGATTCAC | 271 |
| 7 | GTGAGCTTTGCCACCTAAAC | TGAGAGTCACCGTGAAGTAG | 280 |
| 8 | ACCAATTAGCAGACTTGCCG | CTACAGCAATTCTCTTGAG | 264 |
| 9 | CTCAAGAGAATTGCTGTAG | AGGACCGTATGCTTGTTCAC | 326 |
| 10a | TTCCACATACTTTGCGCCCTTC | GCTGTCTTCAGATTCCGA | 235 |
| 10b | CAGAAAGAACAGTCTGGAG | CTCTGAAAGCATTGTGCCA | 256 |
| 11a | CCACATGTCCAATGAC | CACGAACAGAGAGTCTCTTC | 296 |
| 11b | TGATGAGCACAGCACCTTTG | CACCAGTCACAACTCTCTTC | 281 |
| 12 | CTTTGGGCTTTGCTGCTTTC | AAGTAACTGTGACGCAGGAC | 222 |
| 13a | CCTCCAGCAGATTAACCCAT | CAGGTCAACAAATGGGTCCA | 268 |
| 13b | ACACCTTGTCAACCTGGTTG | GATGTCAAGATATACATGGCC | 258 |
| 14 | CCCGTGTTTCAAGAGTATTTGCTC | GCTTATGAACACTCCCAG | 252 |
| 15a | GCAGAGCATTAACACTGTTC | AGCGTGGGAGTTCACAATCA | 241 |
| 15b | GCATGCAGCTCTTTGGTAAG | CCCTTCAGTTGAACACAC | 299 |
| 16a | CCTGTTTTTCCTGCTGTGTTTC | GCCACTAGTAGTTCCATTTCCGTC | 336 |
| 16b | GACAGCTGTATTTCCAACC | AACAGGAAGGAAACACGC | 346 |
| 17 | CTGACCTTTACCAAAGCGGA | GAGGATACTCAAGACCAC | 318 |
| 18 | TGAATCTCCCACCAACAC | GAGTGGATCATGCATCACCT | 252 |
| 19 | CTTAGGCACCTGATAAGAGC | AAAGCAGCAAAGTGCAGC | 302 |
| 20 | CATTGCATAGAGCAAGGC | GGTACAAAGTGTCAGTCTGCTCTC | 263 |
| 21a | TTTCCTTCTCATCCTGTGCC | CTGGCAGTTTGATTGCTCTC | 240 |
| 21b | AGCGTGGTCAACAACTACAG | GCCATTCTAACAGGTGGA | 217 |
| 22 | GCCCCAAAAGTGAATAC | GCGCCAATTTCCCTCTAACTAGAC | 224 |
| 23 | GGGCCCAGAGATTAAAACATGC | CAGAGCAAGGATGAAG | 272 |
| 24 | GAATGAAATGTGGGAGCC | TTCGGGCTGTGAAACGGTTA | 266 |
| 25a | TTACCTCAGCTCTCCAATCACTGG | TGGTCATCGGTTTCCACCAT | 292 |
| 25b | TCATCTGCCTTAACATGGTC | GGGAGTTTGGGATGAATG | 311 |
| 26a | GTACCTAACTGTCCTGTTCAC | TAAACAACGCAGGAAGGGAC | 270 |
| 26b | CACGCTGCTCTTTGCTTTGA | GATCTTTGTCAGGGTCACAG | 269 |
| 26c | GGATGGATTGCTAGCACCTA | TCGCATCGGGATCAAACTTC | 281 |
| 26d | AGCCTCTGAGTGAGGATGAC | TCCATCTGTATTCGAAGGGC | 277 |
| 26e | GTGAGAGTGGAGAGATGGAT | TATCATACGAGGGTGGAGAC | 330 |
| 26f | AACCGATATGACGCCTTCCA | GGTCTCTGTCTTGTTATAGGC | 288 |

Note:
Primer sequences are listed 5′ to 3′. Due to the large size of exons 1, 6, 10, 11, 13, 15, 16, 21, 25 and 26, the exons were split into two or more overlapping amplicons. The neonatally expressed exon 5 is represented as exon 5n.

### REFERENCES

References cited herein are listed on the following pages, and are incorporated herein by this reference.
Annegers, J F. (1996). The treatment of epilepsy: Principles and practice. Second Edition. (Wyllie E (Ed) Williams and Wilkins).
Berkovic, SF. et al. (2004). Ann. Neurol. 55: 550-557.
Commission on Classification and Terminology of the International League against Epilepsy. (1989). Epilepsia 30: 389-399.
Gardiner, M. (2000). J. Neurol. 247: 327-334.
Guipponi, M. Rivier, F. Vigevano, F. Beck, C. Crespel, A. Echenne, B. Lucchini, P. Sebastianelli, R. Baldy-Moulinier, M and Malafosse, A. (1997). Hum. Mol. Genet. 6: 473-477.
Heron, S E. Crossland, KM. Andermann, E. Phillips, H A. Hall, A J. Bleasel, A. Shevell, M. Mercho, S. Seni, M. Guiot, M. Mulley, J C. Berkovic, SF. Scheffer, I E. (2002). Lancet 360:851-52.
Kaplan, R E. and Lacey, D J. (1983). Am. J. Med. Genet. 16: 595-599.
Maxam, A M. and Gilbert, W. (1977). Proc. Natl. Acad. Sci. USA 74: 560-564.
Plouin, P. (1994). In: Malafosse, A. Genton, P. Hirsch, E. Marescaux, C. Broglin, D. and Bernasconi, R. eds. Idiopathic generalised epilepsies: clinical, experimental, and genetic aspects. London: John Libbey and Company. 39-44.
Sanger, F. Nicklen, S. and Coulson, A R. (1977). Proc. Natl. Acad. Sci. USA 74: 5463-5467.
Sutton, G C. (1990). The principles and practice of medical genetics. Second Edition. (Churchill Livingstone, N.Y.).
Szepetowski, P. Rochette, J. Berquin, P. Piussan, C. Lathrop, G M. And Monaco, A P. (1997). Am. J. Hum. Genet. 61: 889-898.
Wyman, A R. and White, R. (1980). Proc. Natl. Acad. Sci. 77: 6754-6758.

1. A method for the diagnosis of a neonatal or infantile epilepsy syndrome as BFNIS in a patient with seizure onset in the first year of life, comprising testing for the presence of an alteration in the SCN2A gene, including in a regulatory region of the gene, in a patient sample, and establishing a diagnosis which will indicate a high probability of BFNIS when an SCN2A alteration is detected or establishing a diagnosis which will indicate a low probability of BFNIS when an SCN2A alteration is not detected.

2. A method as claimed in claim 1 wherein a diagnosis which will indicate a very high probability of BFNIS is established where the SCN2A alteration is known to be BFNIS associated.

3. A method as claimed in claim 1 wherein a diagnosis which will indicate a very high probability of BFNIS is established where the SCN2A alteration is present in the affected parent or relatives of the patient.

4. A method as claimed in claim 1 wherein a diagnosis which will indicate a very high probability of BFNIS is established where the SCN2A alteration is a missense mutation.

5. A method as claimed in claim 1 comprising performing one or more assays to test for the presence of an SCN2A alteration and to identify the nature of the alteration.

6. A method as claimed in claim 1 comprising:
(1) performing one or more assays to test for the presence of an alteration in the SCN2A gene of the patient; and, if the results indicate the presence of an alteration in the SCN2A gene,
(2) performing one or more assays to identify the nature of the SCN2A alteration.

7. A method as claimed in claim 1 further comprising testing for the presence of an alteration in the KCNQ2 and/or KCNQ3 genes, including in the regulatory regions of the genes, in a patient sample, and establishing a diagnosis which will indicate a high probability of BFNS when a KCNQ2 or KCNQ3 alteration is detected or establishing a diagnosis which will indicate a likelihood of BFIS when a KCNQ2 or KCNQ3 alteration is not detected.

8. A method for the diagnosis of a neonatal or infantile epilepsy syndrome as one of BFNIS, BFNS or BFIS in a patient with seizure onset in the first year of life comprising:
(1) (a) testing for the presence of an alteration in the SCN2A gene, including in a regulatory region of the gene, in a patient sample; and/or
(b) testing for the presence of an alteration in the KCNQ2 and/or KCNQ3 genes, including in regulatory regions of the genes, in the patient sample; and
(2) (a) establishing a diagnosis which will indicate a high probability of BFNIS when an SCN2A alteration is detected;
(b) establishing a diagnosis which will indicate a high probability of BFNS when a KCNQ2 or KCNQ3 alteration is detected; or
(c) establishing a diagnosis which will indicate a likelihood of BFIS when an SCN2A, KCNQ2 or KCNQ3 alteration is not detected.

9. A method as claimed in claim 8 comprising performing one or more assays to test for the presence of an SCN2A, KCNQ2 and/or KCNQ3 alteration and to identify the nature of the alteration.

10. A method as claimed in claim 8 comprising:
(1) performing one or more assays to test for the presence of an alteration in the SCN2A, KCNQ2 and/or KCNQ3 genes of the patient; and, if the results indicate the presence of an alteration in any one of these genes,
(2) performing one or more assays to identify the nature of the alteration.

11. A method as claimed in claim 4 wherein one of the assays is a DNA hybridisation assay.

12. A method as claimed in claim 11 wherein an SCN2A, KCNQ2 or KCNQ3 gene probe, an SCN2A, KCNQ2 or KCNQ3 exon-specific probe, or an SCN2A, KCNQ2 or KCNQ3 allele specific probe is hybridized to genomic DNA isolated from said patient.

13. A method as claimed in claim 1 wherein one of the assays is high performance liquid chromatography.

14. A method as claimed in claim 4 wherein one of the assays is an electrophoretic assay.

15. A method as claimed in claim 4 wherein the sample DNA to be tested is quantitatively amplified for at least one exon of the SCN2A, KCNQ2 or KCNQ3 genes to produce amplified fragments and the length of the amplification products for each amplified exon is compared to the length of the amplification products obtained when a wild-type SCN2A, KCNQ2 or KCNQ3 gene is amplified using the same primers, whereby differences in length between an amplified sample exon and the corresponding amplified wild-type exon reflect the occurrence of a truncating alteration in the sample SCN2A, KCNQ2 or KCNQ3 gene.

16. A method as claimed in claim 4 wherein one of the assays incorporates DNA amplification using SCN2A, KCNQ2 or KCNQ3 allele specific oligonucleotides.

17. A method as claimed in claim 4 wherein one of the assays is SSCP analysis.

18. A method as claimed in claim 4 wherein one of the assays is RNase protection.

19. A method as claimed in claim 4 wherein one of the assays is DGGE.

20. A method as claimed in claim 4 wherein one of the assays is an enzymatic assay.

21. A method as claimed in claim 20 wherein said assay incorporates the use of MutS.

22. A method as claimed in claim 4 wherein one of the assays examines the electrophoretic mobility of the SCN2A, KCNQ2 or KCNQ3 proteins of the patient.

23. A method as claimed in claim 4 wherein one of the assays is an immunoassay.

24. A method as claimed in claim 4 wherein one of the assays is DNA sequencing.

25. A method for testing patients for BFNIS-associated mutations in the SCN2A gene comprising the steps of:
a) quantitatively amplifying at least one exon of the SCN2A gene from a body sample of each patient to produce amplified fragments;
b) comparing the properties of the amplified fragments to standard values based upon the fragments produced by amplification of the same exon in a non-mutant SCN2A gene; and
c) determining the nucleic acid sequence of each exon identified in b) that has different properties in the patient compared to the corresponding non-mutant SCN2A exon.

26. A method for testing patients for BFNIS-associated mutations in the SCN2A gene comprising the steps of:
a) quantitatively amplifying, from a body sample of each patient at least one exon of the SCN2A gene using primers complementary to intron regions flanking each amplified exon;
b) comparing the length of the amplification products for each amplified exon to the length of the amplification products obtained when a wild-type SCN2A gene is amplified using the same primers, whereby differences in length between an amplified sample exon and the corresponding amplified wild-type exon reflect the occurrence of a truncating mutation in the sample SCN2A gene; and
c) determining the nucleic acid sequence of each exon identified in b) to contain a truncating mutation.

27. A method for testing patients for BFNIS-associated mutations in the SCN2A gene comprising the steps of:
a) quantitatively amplifying, from a body sample of each patient at least one exon of the SCN2A gene using primers complementary to intron regions flanking each amplified exon;
b) hybridising the fragments from a) with fragments produced by amplification of the same exon in a non-mutant SCN2A gene;
c) determining the nucleic acid sequence of each patient exon identified in b) that either does not hybridise to corresponding wild-type fragments or forms a mismatched heteroduplex.


SEQUENCE LISTING (84 sequences)

SEQ ID NO: 1 - DNA, 20 bp, Homo sapiens
acaggaagttaggtgtggtc

SEQ ID NO: 2 - DNA, 16 bp, Homo sapiens
gagaagcatcacagag

SEQ ID NO: 3 - DNA, 20 bp, Homo sapiens
tgctgtatctcagtgctcag

SEQ ID NO: 4 - DNA, 20 bp, Homo sapiens
tcatcatcctcatccttgcg

SEQ ID NO: 5 - DNA, 16 bp, Homo sapiens
gctaagagacccaaac

SEQ ID NO: 6 - DNA, 20 bp, Homo sapiens
taggcagtgaaggcaacttg

SEQ ID NO: 7 - DNA, 18 bp, Homo sapiens
ggcactattttacagggc

SEQ ID NO: 8 - DNA, 20 bp, Homo sapiens
cataacattgccaaccacag

SEQ ID NO: 9 - DNA, 18 bp, Homo sapiens
tggtgaaggcatggtagt

SEQ ID NO: 10 - DNA, 20 bp, Homo sapiens
attgaggaggtctcaaggtg

SEQ ID NO: 11 - DNA, 18 bp, Homo sapiens
accaacctggaagtgtct

SEQ ID NO: 12 - DNA, 20 bp, Homo sapiens
atagtataggctcccaccag

SEQ ID NO: 13 - DNA, 20 bp, Homo sapiens
aggccccttatatctccaac

SEQ ID NO: 14 - DNA, 20 bp, Homo sapiens
tagcaacaaggcttctgcac

SEQ ID NO: 15 - DNA, 20 bp, Homo sapiens
gatgaaagaccaaggaagac

SEQ ID NO: 16 - DNA, 20 bp, Homo sapiens
tggagatataaggggcctag

SEQ ID NO: 17 - DNA, 18 bp, Homo sapiens
ttccaggacaagctcatg

SEQ ID NO: 18 - DNA, 21 bp, Homo sapiens
ggaagaattatctggaggcca

SEQ ID NO: 19 - DNA, 20 bp, Homo sapiens
ttgttcatgggcaacctacg

SEQ ID NO: 20 - DNA, 20 bp, Homo sapiens
gtctaagtcacttgattcac

SEQ ID NO: 21 - DNA, 20 bp, Homo sapiens
gtgagctttgccacctaaac

SEQ ID NO: 22 - DNA, 20 bp, Homo sapiens
tgagagtcaccgtgaagtag

SEQ ID NO: 23 - DNA, 20 bp, Homo sapiens
accaattagcagacttgccg

SEQ ID NO: 24 - DNA, 19 bp, Homo sapiens
ctacagcaattctcttgag

SEQ ID NO: 25 - DNA, 19 bp, Homo sapiens
ctcaagagaattgctgtag

SEQ ID NO: 26 - DNA, 20 bp, Homo sapiens
aggaccgtatgcttgttcac

SEQ ID NO: 27 - DNA, 22 bp, Homo sapiens
ttccacatactttgcgcccttc

SEQ ID NO: 28 - DNA, 18 bp, Homo sapiens
gctgtcttcagattccga

SEQ ID NO: 29 - DNA, 19 bp, Homo sapiens
cagaaagaacagtctggag

SEQ ID NO: 30 - DNA, 19 bp, Homo sapiens
ctctgaaagcattgtgcca

SEQ ID NO: 31 - DNA, 16 bp, Homo sapiens
ccacatgtccaatgac

SEQ ID NO: 32 - DNA, 20 bp, Homo sapiens
cacgaacagagagtctcttc

SEQ ID NO: 33 - DNA, 20 bp, Homo sapiens
tgatgagcacagcacctttg

SEQ ID NO: 34 - DNA, 20 bp, Homo sapiens
caccagtcacaactctcttc

SEQ ID NO: 35 - DNA, 20 bp, Homo sapiens
ctttgggctttgctgctttc

SEQ ID NO: 36 - DNA, 20 bp, Homo sapiens
aagtaactgtgacgcaggac

SEQ ID NO: 37 - DNA, 20 bp, Homo sapiens
cctccagcagattaacccat

SEQ ID NO: 38 - DNA, 20 bp, Homo sapiens
caggtcaacaaatgggtcca

SEQ ID NO: 39 - DNA, 20 bp, Homo sapiens
acaccttgtcaacctggttg

SEQ ID NO: 40 - DNA, 21 bp, Homo sapiens
gatgtcaagatatacatggcc

SEQ ID NO: 41 - DNA, 25 bp, Homo sapiens
cccgtgtttcaagagtatttgctcg

SEQ ID NO: 42 - DNA, 18 bp, Homo sapiens
gcttatgaacactcccag

SEQ ID NO: 43 - DNA, 20 bp, Homo sapiens
gcagagcattaacactgttc

SEQ ID NO: 44 - DNA, 20 bp, Homo sapiens
agcgtgggagttcacaatca

SEQ ID NO: 45 - DNA, 20 bp, Homo sapiens
gcatgcagctctttggtaag

SEQ ID NO: 46 - DNA, 18 bp, Homo sapiens
cccttcagttgaacacac

SEQ ID NO: 47 - DNA, 22 bp, Homo sapiens
cctgtttttcctgctgtgtttc

SEQ ID NO: 48 - DNA, 24 bp, Homo sapiens
gccactagtagttccatttccgtc

SEQ ID NO: 49 - DNA, 19 bp, Homo sapiens
gacagctgtatttccaacc

SEQ ID NO: 50 - DNA, 18 bp, Homo sapiens
aacaggaaggaaacacgc

SEQ ID NO: 51 - DNA, 20 bp, Homo sapiens
ctgacctttaccaaagcgga

SEQ ID NO: 52 - DNA, 18 bp, Homo sapiens
gaggatactcaagaccac

SEQ ID NO: 53 - DNA, 18 bp, Homo sapiens
tgaatctcccaccaacac

SEQ ID NO: 54 - DNA, 20 bp, Homo sapiens
gagtggatcatgcatcacct

SEQ ID NO: 55 - DNA, 20 bp, Homo sapiens
cttaggcacctgataagagc

SEQ ID NO: 56 - DNA, 18 bp, Homo sapiens
aaagcagcaaagtgcagc

SEQ ID NO: 57 - DNA, 18 bp, Homo sapiens
cattgcatagagcaaggc

SEQ ID NO: 58 - DNA, 24 bp, Homo sapiens
ggtacaaagtgtcagtctgctctc

SEQ ID NO: 59 - DNA, 20 bp, Homo sapiens
tttccttctcatcctgtgcc

SEQ ID NO: 60 - DNA, 20 bp, Homo sapiens
ctggcagtttgattgctctc

SEQ ID NO: 61 - DNA, 20 bp, Homo sapiens
agcgtggtcaacaactacag

SEQ ID NO: 62 - DNA, 18 bp, Homo sapiens
gccattctaacaggtgga

SEQ ID NO: 63 - DNA, 17 bp, Homo sapiens
gccccaaaagtgaatac

SEQ ID NO: 64 - DNA, 24 bp, Homo sapiens
gcgccaatttccctctaactagac

SEQ ID NO: 65 - DNA, 22 bp, Homo sapiens
gggcccagagattaaaacatgc

SEQ ID NO: 66 - DNA, 16 bp, Homo sapiens
cagagcaaggatgaag

SEQ ID NO: 67 - DNA, 18 bp, Homo sapiens
gaatgaaatgtgggagcc

SEQ ID NO: 68 - DNA, 20 bp, Homo sapiens
ttcgggctgtgaaacggtta

SEQ ID NO: 69 - DNA, 24 bp, Homo sapiens
ttacctcagctctccaatcactgg

SEQ ID NO: 70 - DNA, 20 bp, Homo sapiens
tggtcatcggtttccaccat

SEQ ID NO: 71 - DNA, 20 bp, Homo sapiens
tcatctgccttaacatggtc

SEQ ID NO: 72 - DNA, 18 bp, Homo sapiens
gggagtttgggatgaatg

SEQ ID NO: 73 - DNA, 21 bp, Homo sapiens
gtacctaactgtcctgttcac

SEQ ID NO: 74 - DNA, 20 bp, Homo sapiens
taaacaacgcaggaagggac

SEQ ID NO: 75 - DNA, 20 bp, Homo sapiens
cacgctgctctttgctttga

SEQ ID NO: 76 - DNA, 20 bp, Homo sapiens
gatctttgtcagggtcacag

SEQ ID NO: 77 - DNA, 20 bp, Homo sapiens
ggatggattgctagcaccta

SEQ ID NO: 78 - DNA, 20 bp, Homo sapiens
tcgcatcgggatcaaacttc

SEQ ID NO: 79 - DNA, 20 bp, Homo sapiens
agcctctgagtgaggatgac

SEQ ID NO: 80 - DNA, 20 bp, Homo sapiens
tccatctgtattcgaagggc

SEQ ID NO: 81 - DNA, 20 bp, Homo sapiens
gtgagagtggagagatggat

SEQ ID NO: 82 - DNA, 20 bp, Homo sapiens
tatcatacgagggtggagac

SEQ ID NO: 83 - DNA, 20 bp, Homo sapiens
aaccgatatgacgccttcca

SEQ ID NO: 84 - DNA, 21 bp, Homo sapiens
ggtctctgtcttgttataggc